(19) United States
(12) Patent Application Publication
(10) Pub. No.: US 2022/0226183 A1
(43) Pub. Date: Jul. 21, 2022

(54) A SELF DRIVEN REHABILITATION DEVICE AND METHOD THEREOF

(71) Applicant: OSIND MEDI TECH PRIVATE LIMITED, Manipal (IN)

(72) Inventors: Venkateswara Subramania Venkatesan, Manipal (IN); Mohammad Samheel, Udupi (IN); Anil K. Bhat, Manipal (IN)

(21) Appl. No.: 17/617,119

(22) PCT Filed: Jun. 4, 2020
(86) PCT No.: PCT/IB2020/055273

§ 371 Date: Dec. 7, 2021

(30) Foreign Application Priority Data
Jun. 7, 2019 (IN) 201941022639

(51) Int. Cl. A61H 1/02; A61F 5/058; A61B 5/11; A61B 5/00; G16H 20/30; G16H 40/63

(52) U.S. Cl. CPC A61H 1/0288; A61F 5/05866; A61B 5/1125; A61B 5/6806; A61B 5/4836; G16H 20/30; A61H 2201/5061; A61H 2201/1635; A61H 2201/165; A61H 2201/501; A61H 2201/1207; A61H 2201/5097; G16H 40/63

(57) ABSTRACT

The present invention provides a compact, wearable and portable self driven rehabilitation device which is cost effective, light weight, portable, easy to use and customizable to fit as needed. It provides real-time mirroring effect for better visual feedback.

### FIELD OF THE INVENTION

The present invention provides a self driven Rehabilitation Device and its method of operation, useful in stroke and neuro-muscular deficit patients.

### BACKGROUND OF THE INVENTION

The following background discussion includes information that may be useful in understanding the present invention. It is not an admission that any of the information provided herein is prior art or relevant to the presently claimed invention, or that any publication specifically or implicitly referenced is prior art.

Stroke victims often lose proper function of at least one hand and fingers, experiencing delays in gripping and releasing ability, according to Science Daily. The symptoms arise from the effects of strokes on fine motor control in the brain, which regulates movement through muscles, the skeleton and neurological messages, the American Heart Association reports. Strokes often cause at least temporary paralysis on one side of the body, including hands and fingers. The side of the body affected by strokes depends on the side of the brain in which strokes occur, with left brain strokes affecting the right side of the body and right brain strokes affecting the left side, according to the Brain Foundation. The literature states that Hand and finger exercises, movement and physical therapy help stroke victims relearn the use of fine motor skills. CN102387760B discloses kind of wearable action-assist device comprising motion assisting glove (20) having a wearer's finger inserted into the finger insertion portion (21), a drive unit (40), arranged in the auxiliary operation glove back (20) side of the driving finger inserting portion (21), a linear member (50), inserted along the portion of the finger (21) extending in the direction of the drive unit is configured to the drive force to the finger insertion section (21), biological signal detecting unit (60), for detecting the finger movement of the wearer's biosignal, a control unit (70).

US20190021929 discloses a method of rehabilitation using an actuator type that includes a movement mechanism capable of applying a force that interacts with a motion of a patient's limb in a volume of at least 30 cm in diameter, in at least three degrees of freedom of motion of the actuator and capable of preventing substantial motion in any point in any direction in said volume, in which a same movement mechanism is used at two different places of rehabilitation.

US2019105524 discloses a rehabilitation and mobility improvement apparatus (RMIA) comprising a garment, a flexible connection member and an appendage support device, wherein said flexible connection member is connected to said garment on a first end and is connected to said appendage support device on a second end.

U.S. Pat. No. 9,326,909 discloses a therapeutic device for improving voluntary control of paretic muscles in a patient extremity. The therapeutic device is designed to be portable and may be strapped onto a patient's wrist or ankle. The device employs a plurality (Continued) of micro-motors configured to deliver vibratory sensations to a patient extremity as somatosensory inputs. Each micro motor is dimensioned to reside on a patient's respective finger or along their foot. However US '909 fails to provide the feature of mobilization. It delivers vibratory sensations to a patient extremity but no actual movement is being performed.

CN105496728A discloses a soft-bodied robot glove for hand movement function recovery. The soft-bodied robot glove comprises a decision processing module, a driving module, a flexible exoskeleton glove and a Bowden cable. The decision processing module is electrically connected with the driving module. One end of the Bowden cable is connected with the finger end of the flexible exoskeleton glove, and the other end of the Bowden cable is connected to a winch of the driving module. However, CN '728 fails to disclose a modular connection which could help user to select the finger to be mobilized. The system disclosed by CN '728 is highly complex and not portable. It uses the driving module arranged on the single line.

The conventional devices are normally based on passive mobilization therapy and are not the self-driven devices with real-time mirroring effect for better/enhanced visual feedback. The present Invention addresses this technical problem and provides a highly efficient self driven rehabilitation device and method thereof.

### Objective of the Invention

The primary object of the present invention is to overcome the drawback associated with prior art.

Another object of the present invention is to provide a self driven Rehabilitation Device useful in stroke and neuro-muscular deficit patients.

Yet another object of the present invention is to provide a self-driven Rehabilitation device with real-time minoring effect for better visual feedback as compared to the existing devices which are normally based on passive mobilization therapy.

Yet another object of the present invention is to provide an automatic, portable, customizable, compact, light weight, portable, easy to use self driven Rehabilitation Device.

Yet another object of the present invention is to provide a wearable, highly efficient and cost effective self driven Rehabilitation Device.

Yet another object of the present invention is to provide a method of operation of self driven Rehabilitation Device useful in stroke and neuro-muscular deficit patients.

### SUMMARY OF THE INVENTION:

In an aspect of the Invention, there is provided a customizable and light weight self driven rehabilitation device with real time mirroring effect for mobilizing an affected/paralyzed hand of a user comprising:
a pair of glove/s comprising atleast a sensor glove, adapted to be worn in the normal functioning hand of the user, and a modular motorized glove, adapted to be worn in said paralyzed hand of the user;
wherein said sensor glove is connected with the modular motorized glove through a communication module for active/passive mobilization of the paralyzed hand through real time mirroring effect;
wherein sensor glove, provided with an easy to slide system for the fingers, comprises:
   A. atleast a sensing module comprising plurality of flex sensors for sensing movement of fingers based on the change in resistance;
   B. a control/circuit box comprising a communication module for collecting the data from sensing module and transmitting to the motorized glove;
   C. a clincher connector/s connects the sensing module with the control/circuit box; and
   D. a filter circuit adapted remove the noise signal between the sensing module and control box;
   E. a powered supply means comprising a battery for powering the sensor module and control module;

wherein the modular motorized glove, is provided with a drive mechanism adapted to be positioned on the arm of a user, comprises:
A. plurality of fingertip cap/s, configured to be worn on to the finger tips, attached to a tensioning string;
B. motorized unit, configured to be placed in the arm of user, comprising:
   i) plurality of motors for allowing the independent control of the finger/s of the paralyzed hand wherein each motor is connected to individual strings for extension;
   ii) a motor driver unit/mechanism to drive said plurality of motors wherein the driver unit/mechanism is adapted to move towards back on the arm for increased range of motion;
   iii) a microcontroller unit with communication module configured to receive the data transmitted from said sensor glove to accordingly control the said motorized unit;
      wherein the strings are connected through the modular connector acting like a bridge between the motorized unit and glove;
   iv) a power supply means to power the motor driver unit and the microcontroller unit;
   v) plurality of metal hooks connected to a rhodium plated cloum comprising a barrel swivel clasps/connectors wherein the barrel swivel connectors connects the glove to the motorized unit;
   vi) atleast a closure unit/mechanism being placed at the top of said motorized unit with a bridge in between, said closure unit/mechanism comprises the characterizing feature of a dial which allows the torque to be adjusted by its rotation for obtaining range of mobilization;
   vii) connecting means comprising modular connector for selecting the finger to be mobilized and for the activation of active/passive mode of therapy based on requirement;
where said device optionally comprises wrist splint for restricting the wrist movement.

In another aspect of the Invention, there is provided a method of operation of the customizable and light weight self driven rehabilitation device with real time mirroring effect for mobilizing an affected/paralyzed hand of a user, as described above, comprising the step/s of:
a) wearing the sensor glove in the normal hand of a user followed by sensing the motion of the finger/s by the sensor module wherein the sensor module sends the sensed/detected range of motion of finger to the control box for further transmission to the motorized glove through the communication module;
b) receiving the transmitted signal by the motorized unit, placed at the arm of the user, of the motorized glove, followed by the activation of the control unit configured to activate the drive unit to drive the plurality of motor/s adapted to mobilize the affected finger/s connected with the tensioning wire through modular connector;
   wherein the closure unit/mechanism being placed at the top of said motorized unit with a bridge in between, said closure unit/mechanism comprises the characterizing feature of a dial which allows the torque to be adjusted by its rotation for obtaining range of mobilization.

### DETAILED DESCRIPTION OF DRAWINGS

To further clarify advantages and features of the present invention, a more particular description of the invention will be rendered by reference to specific embodiments thereof, which is illustrated in the appended drawings. It is appreciated that these drawings depict only typical embodiments of the invention and are therefore not to be considered limiting of their scope. The invention will be described and explained with additional specificity and detail with the accompanying drawings in which:

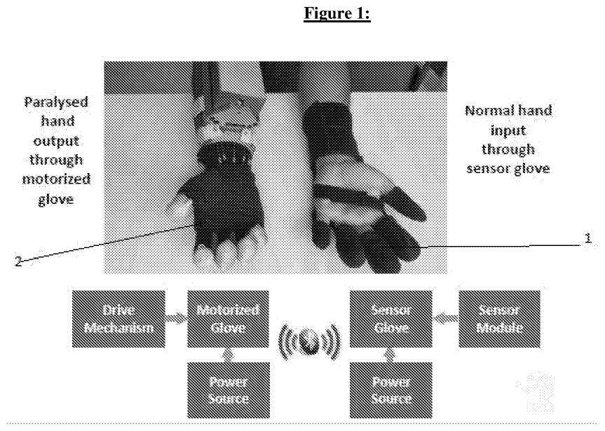

FIG. 1: illustrates functioning of the device according to an embodiment of the invention

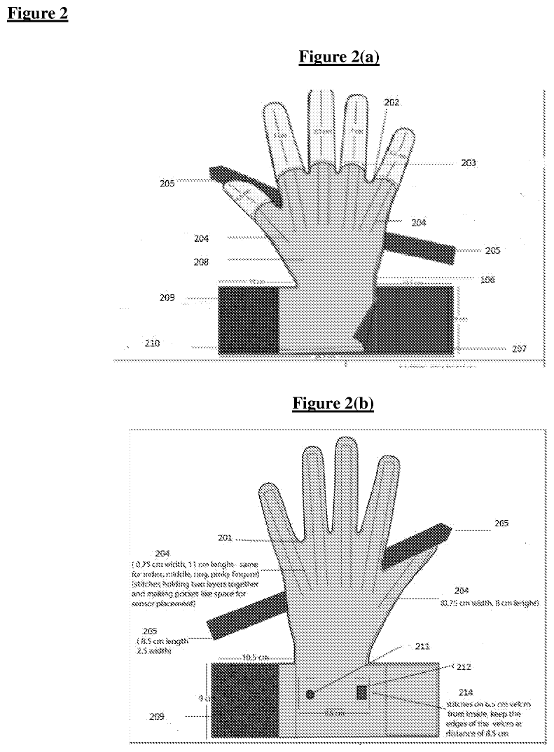

FIG. 2: illustrates (a) front side of the sensor glove (b) back side of the sensor glove

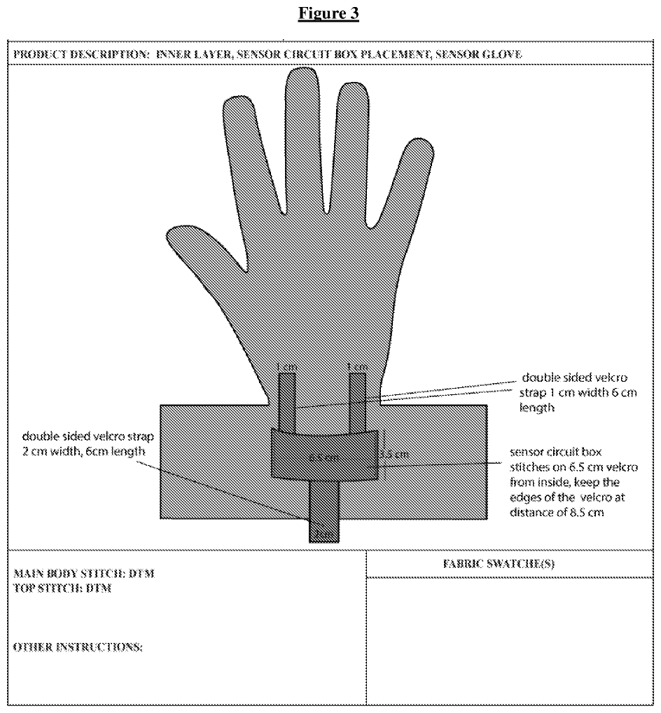

FIG. 3: illustrates inner layer of the sensor glove enclosing sensor circuit box and sensor glove

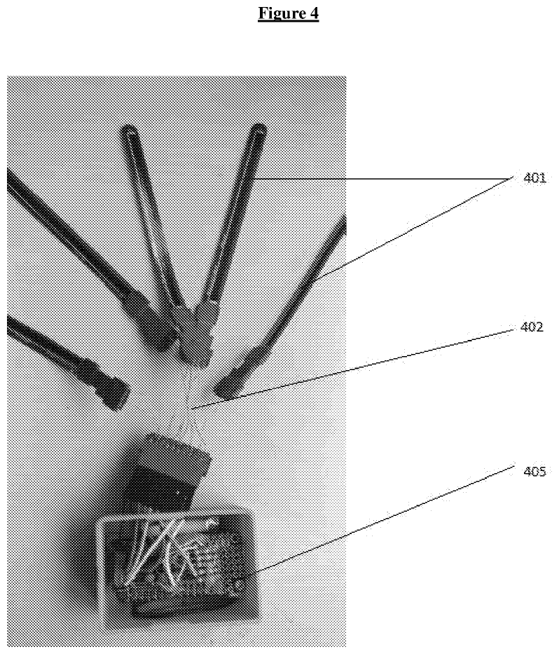

FIG. 4: illustrates components of the device according to an embodiment of the invention

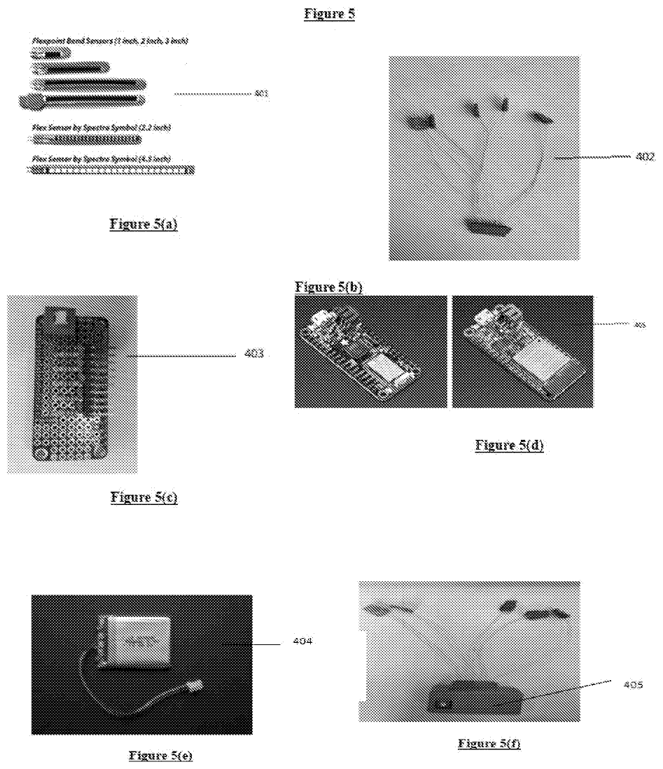

FIG. 5: illustrates components of the sensor glove—5(a) illustrates flex sensors; 5(b) illustrates clincher connectors; 5(c) illustrates filter circuit; 5(d) illustrates BLE module or wi-fi module; 5(e) illustrates battery powering the sensor module; 5(f) illustrates sensor glove circuit box

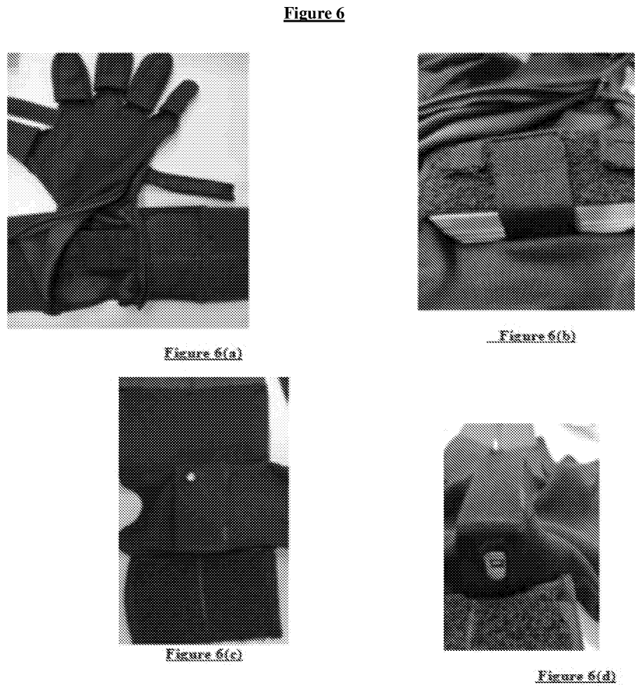

FIG. 6: illustrates the casing design within glove as illustrated in FIG. 6(a), Casing within sensor module in FIG. 6(b), Top view of charging port on and off button in FIG. 6 (c), Side view of charging port ON and OFF button in FIG. 6 (d)

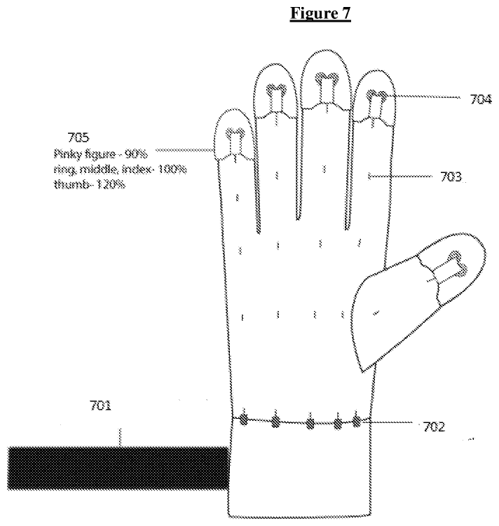

FIG. 7: illustrates front side of the motorized glove

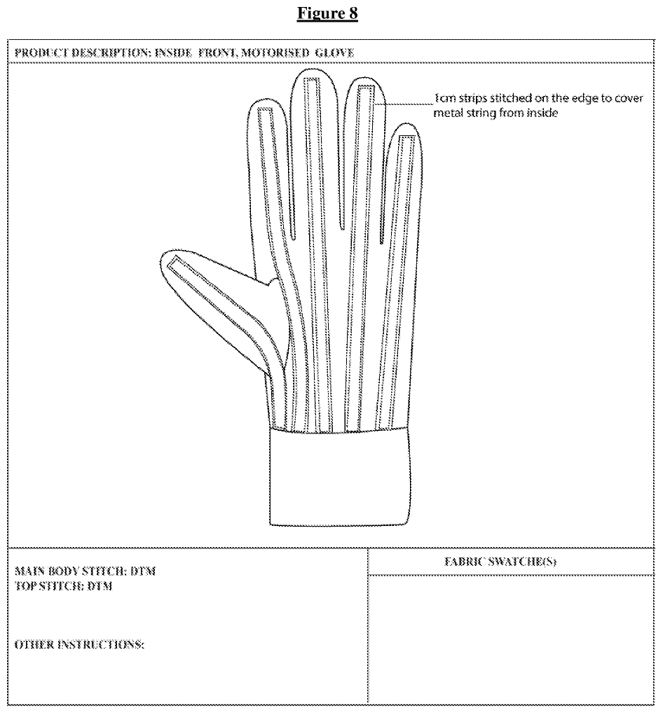

FIG. 8: illustrates inside layer front of the motorized glove where 1 cm strips is stitched on the edge to cover metal string from inside

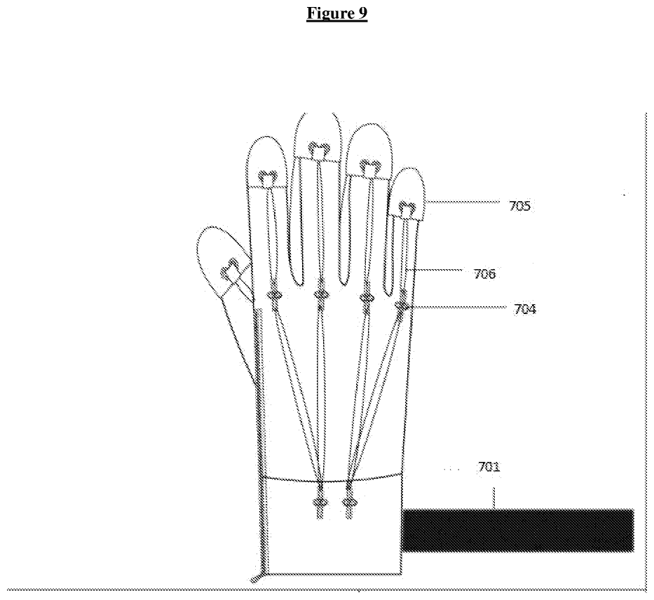

FIG. 9: illustrates back side of the motorized glove showing tensioner location and connections

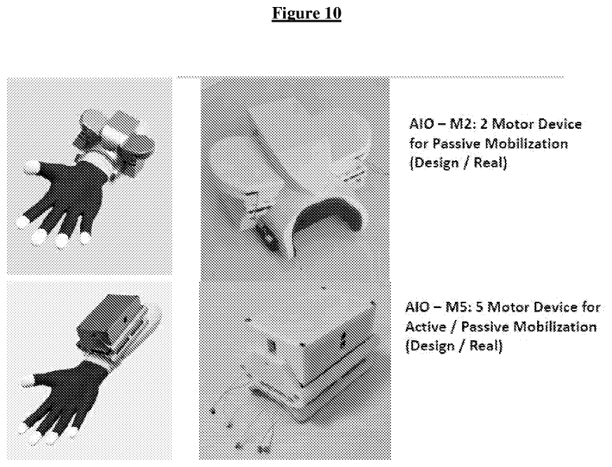

FIG. 10: illustrates functioning of 2 motor device and 5 motor device in accordance to an embodiment of the Invention.

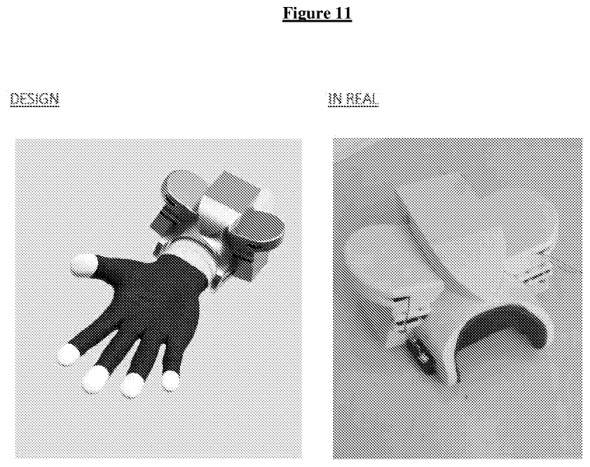

FIG. 11: illustrates two motor device having modular nature

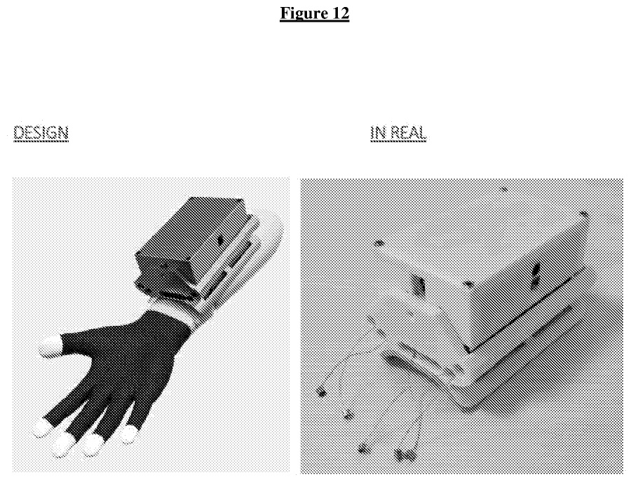

FIG. 12: illustrates functioning of the five motor device

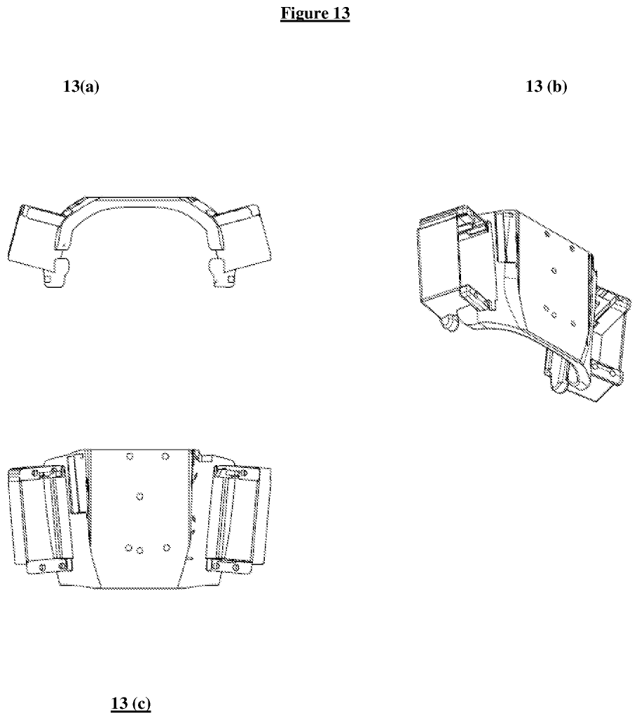

FIG. 13: illustrates an embodiment showing base of the closure unit/echa ism, as provided by the present invention

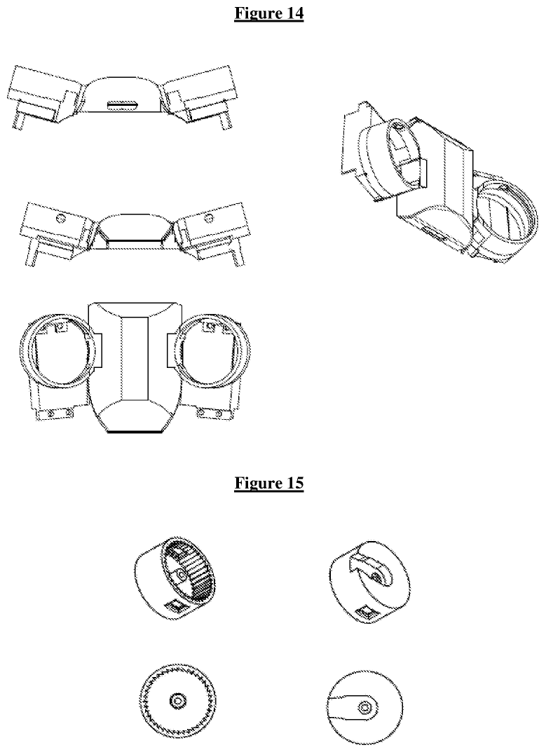

FIG. 14: illustrates another embodiment showing top of the closure unit/mechanism, as provided by the present invention

FIG. 15: illustrates another embodiment showing closure system bridge servo horn attachment

### DETAILED DESCRIPTION:

For the purpose of promoting an understanding of the principles of the invention, reference will now be made to the embodiment illustrated in the drawings and specific language will be used to describe the same. It will nevertheless be understood that no limitation of the scope of the invention is thereby intended, such alterations and further modifications in the illustrated system, and such further applications of the principles of the invention as illustrated therein being contemplated as would normally occur to one skilled in the art to which the invention relates.

It will be understood by those skilled in the art that the foregoing general description and the following detailed description are exemplary and explanatory of the invention and are not intended to be restrictive thereof.

The Invention provides a self driven Rehabilitation Device/system useful in stroke and neuro-muscular deficit patients and its method of operation. The device/system is a self-driven Rehabilitation device with real-time mirroring effect for better visual feedback as compared to the existing devices which are normally based on passive mobilization therapy. The device/system allows smooth and realistic movements. The device/system is helpful for the patients who cannot do regular therapy due to distance between their home and the hospital with a home based rehabilitation program. The present device/system has various technical advantages in terms of being automatic, portable, customizable and compact. The device/system is cost effective, light weight, portable, easy to use and customizable to fit as needed. The system is wearable and highly portable. This allows the therapy to be done from home without the supervision of the doctor/therapist. The user movements can be tracked using remote monitoring for gathering data of usage.

In an embodiment, the customizable and light weight self driven rehabilitation device with real time mirroring effect for mobilizing an affected/paralyzed hand of a user comprises a pair of gloves comprising atleast a sensor glove, adapted to be worn in the normal functioning hand of the user, and a modular motorized glove, adapted to be worn in said paralyzed hand of the user. The sensor glove is connected with the modular motorized glove through a communication module for active/passive mobilization of the paralyzed hand through real time mirroring effect. The sensor glove is provided with an easy to slide system for the fingers.

In an embodiment, the sensor glove comprises following components:
i. atleast a sensing module comprising plurality of flex sensors for sensing movement of fingers based on the change in resistance;
ii. a control/circuit box comprising a communication module for collecting the data from sensing module and transmitting to the motorized glove;
iii. a Clincher connectors connects the sensing module with the control/circuit box; and
iv. a filter Circuit adapted to remove the noise signal between the sensing module and control box;
v. a powered supply means comprising a battery for powering the sensor module and control module,

In an embodiment, the sensor glove comprises atleast 3 layers. The first layer is illustrated in FIG. 2 which consists of part that comes in contact with the hand i.e. dorsal region of palm of the subject. The first layer consists of plurality of Velcro strips for binding the gloves and components that come in contact with the hand of the subject/user The intermediate layer is illustrated in FIG. 3 which houses the sensor module (control box) while the third layer as illustrated in FIG. 2a is configured to position the sensor module and the clincher connectors i.e. the sensor module groves are present for placement and clincher connectors are placed and connected.

The sensor glove uses an easy to slide system for the fingers with lesser cloth material on the palm. The control system is housed inside the glove itself as a sensor box with battery side for easy to wear. The sensors and its wiring is hidden within in the intermediate layer consisting of clincher connector for modular connectivity of sensors.

In an embodiment, the modular motorized glove is provided with a drive mechanism wherein the drive mechanism rests/positioned on the arm of a user. Particularly, the motorized glove is worn on the hand and the drive mechanism is placed on the arm. Battery used is a powerbank which is placed nearby or in pocket.

When in air, or surface, the load will applied on the fingers will be only of the glove which is approximately 50 g. Hence, the device is super lightweight in comparison to the existing products in the market or known otherwise.

The modular motorized glove comprises following components:
i. plurality of fingertip cap/s, configured to be worn on to the finger tips, attached to a tensioning string;
ii. motorized unit, configured to be placed in the arm of user, comprising:
   a. plurality of motors for allowing the independent control of the finger/s of the paralyzed hand wherein each motor is connected to individual strings for extension;
   b. a motor driver unit/mechanism to drive said plurality of motors wherein the driver unit/mechanism is adapted to move towards back on the arm for increased range of motion;
   c. a microcontroller unit with communication module configured to receive the data transmitted from said sensor glove to accordingly control the said motorized unit;
      wherein the strings are connected through the modular connector acting like a bridge between the motorized unit and glove;
   d. a power supply means to power the motor driver unit and the microcontroller unit;
   e. plurality of metal hooks connected to a rhodium plated cloum comprising a barrel swivel clasps/connectors wherein the barrel swivel connectors connects the glove to the motorized unit;
   f. atleast a closure unit/mechanism being placed at the top of said motorized unit with a bridge in between, said closure unit/mechanism comprises the characterizing feature of a dial which allows the torque to be adjusted by its rotation for obtaining range of mobilization;
iii. connecting means comprising modular connector for selecting the finger to be mobilized and for the activation of active/passive mode of therapy based on requirement.

The system optionally comprises wrist splint for restricting the wrist movement. In an embodiment, the device comprises the motor driver unit based on 2 motor or 5 motor operation system. The 2 motor operation system operate/s in plurality of active/passive mode by connecting at least two finger of the hand or its combination thereof. 5 motor operation system operate/s in plurality of modes based on the requirement of mobilization. The 5 motor operation system allows independent movement of each finger facilitating plurality of modes of operation for active/passive mode.

In an embodiment, the motor operation system comprises servo motor system.

In an embodiment, the bridge in the closure unit comprises a servo horn attachment. The closure unit/mechanism comprises a dial at the top. The dial allows the torque to be adjusted by its rotation and helps in setting the appropriate range of motion.

In an embodiment, FIG. 13 shows the base of the motorized unit with two motor systems. The base of the motorized unit is configured to be placed or worn on the arm of the paralyzed hand/therapeutic hand for mobilization. FIGS. 13(a), 13(b) and 13(c) shows the front, side and top view of the base of said motorized unit. The motorized unit can easily be placed on the arm or rest on any surface without affecting the motion.

FIG. 14 shows the top of the motorized unit with two motor systems which is configured to be placed/fitted with the bottom of the motorized unit. The top is adapted to house 2 motor/s, thus making it highly portable and easy to wear. The motor system simply runs with the power bank. The top of the motorized unit comprises a space for adapting a closure system bridge with servo horn attachment as shown in FIG. 15. This closure system comprises a dial at the top which allows the torque to be adjusted by rotating the dial and set appropriate range of motion.

In an embodiment, the sensor glove/s comprises plurality of layer/s wherein the layers comprises:
a) a first layer, configured to consists plurality of Velcro strips for binding the gloves and components that come in contact with the hand of the subject/user;
b) an intermediate layer configured to in-house the sensor and connecting means comprising clincher connector ; and
c) a third layer configured to position the sensor module and the clincher connectors.

In an embodiment, the system consists of two variable changes, as mentioned below, that account for change in range of mobilization.
a) Moving the drive mechanism further back on the arm for increased range; and
b) The Closure system dial which allows the torque to be adjusted by rotating the dial and setting appropriate range of mobilization. This allows the position of the drive mechanism to be unchanged and still benefit from varying the range of mobilization and torque. Both Palm surfaces are free for movement and can move the wrist. If the wrist movement needs to be restricted, the design is made in such a way that it can accommodate a wrist splint under the glove and drive mechanism, to allow maximum finger movement only.

In an embodiment, the plurality of motor comprises two motor for activating atleast two finger/s or five motor system for activating all the five finger/s, as required.

In an embodiment, sensor glove being in communication to the motorized glove provides feedback based on virtual reality environment.

In an embodiment, the sensor glove functions in mode/s comprising:
a. active mode for transmitting position data for mobilizing the finger/s;
b. diagnostic mode for wirelessly tracking the range of motion through a software application;
c. virtual reality gaming mode where the sensor glove tracks the range of motion and wirelessly transmits the data to a software application. The sensor glove is used for Non Immersive or Immersive VR Gaming as a tool for motivation for people who have regained some motion but have to repeat it to keep the functional recovery. The sensor glove has the ability to track the movement (range of motion) as well as it wirelessly transmits data to android app and at the same time acts as controller for the game in Real Time.

In an embodiment, the glove comprises groove/s for the strings to slide through with ease for providing maximum torque.

In an embodiment, the range of motion of the affected limb is remotely monitored by an application software for evaluating the improvement in the user.

In an embodiment, the clincher connectors comprises plastic coating for eliminating the overlap of signals received from the plurality of sensors.

In an embodiment, the modular connector of motorized glove is in connection to the Flexible fingertip cap where the fingertip cap also attaches itself to the tensioning system on the back side to provide passive extension or passive flexion.

In an embodiment, the sensor glove comprises a force sensitive resistor on the fingertip to measure the pinch force for evaluating the improvement in the patient.

In an embodiment referring to the FIG. 1, the self driven rehabilitation system comprises a pair of gloves having atleast a motorized-glove (2) and a sensor glove (1) along with a motorized unit (3).

In an embodiment, the sensor glove (1) is worn on the normal hand and the motorized glove (2) on the affected/paralyzed hand and the motorized unit (3) is worn on the affected arm. The system is effective for mobilization of the hand and allows user to mirror the movements of his normal hand to the affected arm under active mode/active mobilization.

In an embodiment, the movements can be pre-programmed based on the users need under passive mode/passive mobilization.

In an embodiment, the sensor glove (1) communicates with the motorized-glove (2) through communications module like blue tooth to replicate or mirror the activities done on the functional hand.

In an embodiment, the connecting mechanism between said modular and sensor glove is modular.

In an exemplary embodiment, FIGS. 2(a) and 2(b) illustrates front side and back side of the sensor glove respectively.

FIG. 2(a) shows front side of the sensor glove showing following components:
a) Multiple Velcro straps (205,209) having length ranging from 7.5 cm to 9 cm length and width ranging from 2.5 cm to 5 cm;
b) Edge stitch (201) in between the joint of fingers;
c) Multiple Sensors (204) having approximately 0.75 cm width and length ranging from 8 cm to 11 cm
d) Multiple layers (207, 208) i.e. Layer 1 and Layer 2
e) Invisible zip(206)
f) Stitched portion (203) holding two layers together and making pocket like space for sensor placement
g) Single fold stitch (210) having 1 cm length.

FIG. 2(b) shows back side of the sensor glove showing following components:
a) Multiple Velcro straps 205,209), having length ranging from 7.5 to 9 cm length and width ranging from 2.5 cm to 9 cm;
b) Edge stitch(203) in between the joint of fingers;
c) Multiple Sensors (204) having approximately 0.75 cm width and length ranging from 8 cm to 11 cm
d) atleast a button hole (211) stitch-opening for on/off button
e) atleast a button hole stitch-opening for charging port (212)
f) sensor circuit box (214) stitches on 6.5 cm Velcro from inside keeping the edges of the Velcro at distance of 8.5 cm.

In an embodiment, the inner layer of the sensor glove is illustrated in FIG. 3. The inner layer comprises a double sided Velcro strap (1 cm width and 6 cm length), another double sided velcro strap (2 cm width with 6 cm length), sensor circuit box stitched on 6.5 cm Velcro from inside keeping the edges of the Velcro at distance 8.5 cm

FIGS. 4 to 5 shows comprehensive view of the components of the sensor glove. In an embodiment, the sensor glove comprises following components:
a) Flex Sensors (401)
b) Clincher connectors (402)
c) Filter Circuit (403
d) Battery for powering the sensor module (BLE module or wi-fi module) (404)
e) Sensor Glove Circuit Box (405)

In an embodiment, the Flex Sensors (401) senses the movement of fingers based on the change in resistance. The flex sensors comprises flexpoint bend sensors (1 inch, 2 inch, 3 inch), flex sensor by spectra symbol (2.2 inch), flex sensor by spectra symbol (4.5 inch).

In an embodiment, the clincher connectors are used for connection between the Flex Sensors and the Sensor Circuit. The wires connecting these two have a coating of plastic which doesn't allow overlap of the signals. The connection is modular using the clasps connector system. User can choose which finger to mobilize while connecting the clasps. The connecting mechanism is modular where the strings are connected to the Flexible fingertip cap where the Fingertip cap also attaches itself to the tensioning system on the back side to provide extension.

In an embodiment, the filter circuit filters the noise from the signal and provides the location for Switch ON/Off Button. It also acts as modular connecting point for clincher connectors. The filter circuit is based on the microcontroller/Communication channel used.

In an embodiment, the sensor glove comprises Adafruit NRF 51 (BLE module) or ESP 32 (Wi-Fi module). It further comprises microcontroller charging port and communication channel.

In an embodiment, the battery is Li po battery.

In an embodiment, the casing design within glove is illustrated in FIG. 6(a), Casing within sensor module is illustrated in FIG. 6(b), Top view of charging port on and off button is illustrated in FIG. 6(c), Side view of charging port on and off button is illustrated in FIG. 6(d).

In an embodiment, the sensor glove provides a way to be used in Virtual Reality (Both Immersive and Non—Immersive environment) based rehabilitation using games for motivation for repetitive therapy. This allows the patient to use the sensor glove on normal hand and mobilize affected hand while playing a game or it can be used in stroke patients who have partial movement but need motivation to do therapy.

In an embodiment, the sensor glove is combined with motorized glove to give the feedback based on virtual reality environment. As an example: If a user is trying to squeeze a lemon in virtual reality, then the sensor glove senses the movement and feeds the information to allow the motors to simultaneously produce proportional force to be applied in the opposite direction to get the haptic feedback.

The user is able to perform the accurate movement with the help of sensor glove by controlling the movement of the affected arm.

In an embodiment, the sensor glove is useful to detect the Range of Motion of the affected limb when the patients/user come for follow up visits. In such embodiment, the patient can wear this glove on their affected hand and their progress in Range of motion can be remotely monitored using the Android Application for evaluating the improvement in the patient. Referring to FIG. 7, which illustrates front side of the motorized glove (2) showing Velcro straps (701) (11 inches length and 2.5 inches width), rhodium plated column barrel swivel clasps (702), string (703), metal hooks(704), finger tip caps (705).

FIG. 8 illustrates inside layer front of the motorized glove (2) where 1 cm strips is stitched on the edge to cover metal string (703) from inside.

FIG. 9 illustrates back side of the motorized glove showing tensioner location and connections. The figure shows velcro strap of approximately 11 inches, hooks (704) connecting through rubber band tensioners to finger tip cap (705) from back.

In another embodiment, the device comprises a force sensitive resistor on the fingertip to measure the Pinch force for evaluating the improvement in the patient.

In another embodiment, the motorized glove (2) comprises metal hooks (704) connected to the rhodium plated cloum barrel swivel clasps (702) via strings (703). The barrel swivel connectors act as the connecting point between glove and the motorized unit. In an embodiment, the motorized unit (3) comprises 2 motors/5 motors (allowing independent finger of each control) to be placed in the arm of the subject/user/patient, as illustrated in FIG. 10. The barrel swivel connectors (702) act as the connecting point between glove and the motorized unit (3). The unit also comprises microcontroller circuit (BLE/Wi-Fi), dedicated Motor Driver to power the motors. The unit is powered by a simple powerbank which can support the microcontroller at 3.3V and Motors at 5V simultaneously. The modular connectors help for choosing which finger to mobilize and activate active/passive mode of therapy based on requirement.

In an embodiment, the method of operation of the customizable and light weight self driven rehabilitation system with real time mirroring effect for mobilizing an affected/paralyzed hand of a user, as described above, comprises following steps:
a) wearing the sensor glove in the normal hand of a user followed by sensing the motion of the finger/s by the sensor module wherein the sensor module sends the sensed/detected range of motion of finger to the control box for further transmission to the motorized glove through the communication module;
b) receiving the transmitted signal by the motorized unit, placed at the arm of the user, of the motorized glove, followed by the activation of the control unit configured to activate the drive unit to drive the plurality of motor/s adapted to mobilize the affected finger/s connected with the tensioning wire through modular connector.
   The closure unit/mechanism is placed at the top of said motorized unit with a bridge in between. The closure unit/mechanism comprises the dial which allows the torque to be adjusted by its rotation for obtaining range of mobilization.

Advantages of the Self Driven Rehabilitation Device Provided by the Present Invention:
a) The motorized glove is worn on the hand and the drive mechanism is placed on the arm. The user can easily place the arm and rest on any surface without affecting the motion.
b) The motorized glove is designed in such a way that it is modular and it can be used with any of the motorized systems.
c) The device is light weight. When in air or surface, the load applied on the fingers will be only of the glove which is (50 g). Hence, the device is super lightweight in comparison to the available products.
d) The device provides increased range of motion with two variable changes that account for change in range of mobilization as mentioned below:
   i) Moving the drive mechanism further back on the arm for increased range.
   ii) The Closure unit dial allows the torque to be adjusted by its rotation and setting the appropriate range of mobilization. This allows the position of the drive mechanism to be unchanged and still achieves the mobilization by varying the range of mobilization and torque.
e) Both Palm surfaces are free for movement and can move the wrist. If the wrist movement needs to be restricted, the design is made in such a way that it can accommodate a wrist splint under the glove while the drive mechanism allows maximum finger movement only.
f) The modular system includes 2 or 5 motors based on requirement so that the patient doesn't need to bear the extra weight. The connection is modular and is based on the clasp connector system. User can select which finger to mobilize while connecting the clasps.
g) Wrist splint can be added to provide restriction to wrist movement.
h) The device does not just trick the brain, but causes real mobilization at the same time.
i) The device is highly cost effective

The Invention is further described with the help of non-limiting examples:

### EXAMPLE 1

The device, as illustrated in FIG. 11, is a two motor device having modular nature and can operate in Various Modes as mentioned below:
Pinch Mode (Thumb+Index or Thumb+Middle, so on)
Two Fingers connected to one motor for passive mobilization. (So we can connect (Thumb+Middle+Ring+Little finger)for passive mode to mobilize for Grasp mode.
Single finger Flexion/Extension. (Active/Passive)
Selected two finger mobilization. (Active/Passive)

### EXAMPLE 2

The device, as illustrated in FIG. 2, is a five motor device and can operate in various modes based on the requirement by the doctor as every patient has a different requirement of mobilization. Hence the patient/user can train/operate his normal hand in the way his affected hand should and the patient can carry out the activity at home and vary it based on his improvement over time. The device helps in allowing independent movement of each fingers. The device helps in 13 modes of operation (PASSIVE—Pre Programmed) as following:
a. All open all close
b. Counting exercise
c. first exercise
d. Grasping object exercise
e. Handle exercise
f. Picking objects exercise
g. Pinch exercise
h. Pinches sequence exercise
i. Random numbers exercise
j. Single singer random exercise
k. Single finger sequence
l. Wave exercise.
m. Custom exercise for patient.

### EXAMPLE 3

The five Motor device comprises following components:
a) The barrel swivel connectors act as the connecting point between glove and the motorized unit
b) 5 Motors: (Independent control of each finger) , Microcontroller circuit (BLE/Wi-Fi), Dedicated Motor Driver to power the motors
c) Powered by a simple Powerbank which can support the microcontroller at 3.3V and Motors at 5V simultaneously
d) Modular connectors help for choosing which finger to mobilize
e) Active/Passive mode of therapy is available based on requirement
f) Best used for nerve injury cases for better functional recovery
g) Remote monitoring and control.

### EXAMPLE 4

The motor device helps in improving range of motion in tendon injury patients and preventing stiffness arising due to non compliance to therapy recommended to be done at home.

The two motor device comprises following components:
a) 2 motors. This can be connected to 4 fingers simultaneously (2 each) or the fingers can be connected based on Mode chosen. For example: 10 Modes+Cutsom Mode available to tailor make it to suit each patient need, Pinch Mode (Thumb+Index finger)
b) Lightweight , Half the weight of five motor device (only 220 g)
c) Remote monitoring and control.

1. A customizable and light weight self driven rehabilitation device with real time mirroring effect for mobilizing an affected/paralyzed hand of a user comprising:
a pair of glove/s comprising atleast a sensor glove, adapted to be worn in the normal functioning hand of the user, and a modular motorized glove, adapted to be worn in said paralyzed hand of the user;
wherein said sensor glove is connected with the modular motorized glove through a communication module for active/passive mobilization of the paralyzed hand through real time mirroring effect;
wherein sensor glove, provided with an easy to slide system for the fingers, comprises:
A. atleast a sensing module comprising plurality of flex sensors for sensing movement of fingers based on the change in resistance;
B. a control/circuit box comprising a communication module for collecting the data from sensing module and transmitting to the motorized glove;
C. a clincher connector/s connects the sensing module with the control/circuit box; and
D. a filter circuit adapted remove the noise signal between the sensing module and control box;
E. a powered supply means comprising a battery for powering the sensor module and control module;
wherein the modular motorized glove, is provided with a drive mechanism adapted to be positioned on the arm of a user, comprises:
A. plurality of fingertip cap/s, configured to be worn on to the finger tips, attached to a tensioning string;
B. motorized unit, configured to be placed in the arm of user, comprising:
i) plurality of motors for allowing the independent control of the finger/s of the paralyzed hand wherein each motor is connected to individual strings for extension;
ii) a motor driver unit/mechanism to drive said plurality of motors wherein the driver unit/mechanism is adapted to move towards back on the arm for increased range of motion;
iii) a microcontroller unit with communication module configured to receive the data transmitted from said sensor glove to accordingly control the said motorized unit;
wherein the strings are connected through the modular connector acting like a bridge between the motorized unit and glove;
iv) atleast a closure unit/mechanism being placed at the top of said motorized unit with a bridge in between, said closure unit/mechanism comprises the characterizing feature of a dial which allows the torque to be adjusted by its rotation for obtaining range of mobilization;
v) a power supply means to power the motor driver unit and the microcontroller unit;
vi) plurality of metal hooks connected to a rhodium plated cloum comprising a barrel swivel clasps/connectors wherein the barrel swivel connectors connects the glove to the motorized unit;
vii) connecting means comprising modular connector for selecting the finger to be mobilized and for the activation of active/passive mode of therapy based on requirement;
where said system optionally comprises wrist splint for restricting the wrist movement.

2. The device as claimed in claim 1, wherein the motor driver unit comprises 2 motor or 5 motor operation system, wherein the 2 motor operation system operate/s in plurality of active/passive mode by connecting at least two finger of the hand or its combination thereof while the 5 motor operation system operate/s in plurality of modes based on the requirement of mobilization wherein the 5 motor operation system allows independent movement of each finger facilitating plurality of modes of operation for active/passive mode.

3. The device as claimed in claim 1, wherein the motor operation system comprises servo motor system.

4. The device as claimed in claim 1, wherein the bridge in the closure unit comprises a servo horn attachment.

5. The device as claimed in claim 1, wherein the sensor glove/s comprises plurality of layer/s wherein the layers comprises:
a) a first layer, configured to consists plurality of Velcro strips for binding the gloves and components that come in contact with the hand of the subject/user;
b) an intermediate layer configured to in-house the sensor and connecting means comprising clincher connector ; and
c) a third layer configured to position the sensor module and the clincher connectors.

6. The device as claimed in claim 1, wherein the sensor glove being in communication to the motorized glove provides feedback based on virtual reality environment.

7. The device as claimed in claim 1, wherein the sensor glove functions in mode/s comprising:
a. active mode for transmitting position data for mobilizing the finger/s;
b. diagnostic mode for wirelessly tracking the range of motion through a software application;
c. virtual reality gaming mode where the sensor glove tracks the range of motion and wirelessly transmits the data to a software application.

8. The device as claimed in claim 1, wherein said glove comprises groove/s for the strings to slide through with ease for providing maximum torque.

9. The device as claimed in claim 1, wherein the range of motion of the affected limb is remotely monitored by an application software for evaluating the improvement in the user.

10. The device as claimed in a claim 1, wherein the clincher connectors comprises plastic coating for eliminating the overlap of signals received from the plurality of sensors.

11. The device as claimed in claim 1, wherein the modular connector of motorized glove is in connection to the Flexible fingertip cap where the fingertip cap also attaches itself to the tensioning system on the back side to provide passive extension or passive flexion.

12. The device as claimed in a claim 1, wherein the sensor glove comprises a force sensitive resistor on the fingertip to measure the pinch force for evaluating the improvement in the patient.

13. A method of operation of the customizable and light weight self driven rehabilitation device with real time mirroring effect for mobilizing an affected/paralyzed hand of a user, as claimed in claim 1, comprising the step/s of:
a) wearing the sensor glove in the normal hand of a user followed by sensing the motion of the finger/s by the sensor module wherein the sensor module sends the sensed/detected range of motion of finger to the control box for further transmission to the motorized glove through the communication module;
b) receiving the transmitted signal by the motorized unit, placed at the arm of the user, of the motorized glove, followed by the activation of the control unit configured to activate the drive unit to drive the plurality of motor/s adapted to mobilize the affected finger/s connected with the tensioning wire through modular connector;
wherein the closure unit/mechanism being placed at the top of said motorized unit with a bridge in between, said closure unit/mechanism comprises the characterizing feature of a dial which allows the torque to be adjusted by its rotation for obtaining range of mobilization.

14. The method as claimed in claim 13, wherein drive unit comprises 2 motor or 5 motor operation system, wherein the 2 motor operation system operate/s in plurality of active/passive mode by connecting at least two finger of the hand or its combination thereof while the 5 motor operation system operate/s in plurality of modes based on the requirement of mobilization wherein the 5 motor operation system allows independent movement of each finger facilitating plurality of modes of operation for active/passive mode.
